(19) United States
(12) Patent Application Publication
(10) Pub. No.: US 2008/0187979 A1
(43) Pub. Date: Aug. 7, 2008

(54) Method for Preparing Sample Solution and Sample Solution Preparing Apparatus

(76) Inventor: Toshihiro Mori, Saitama (JP)

Correspondence Address: BIRCH STEWART KOLASCH & BIRCH, PO BOX 747, FALLS CHURCH, VA, 22040-0747, US

(21) Appl. No.: 11/793,314

(22) PCT Filed: Jan. 31, 2006
(86) PCT No.: PCT/JP2006/301923

§ 371 Date: Jun. 19, 2007

(30) Foreign Application Priority Data
Jan. 31, 2005 (JP) 2005-022930

(51) Int. Cl. C12N 9/14; C07H 19/00

(52) U.S. Cl. 435/195; 536/22.1

(57) ABSTRACT

A method for preparing a sample solution, which is a step preceding a process for separating and purifying a nucleic acid by extracting a nucleic acid from the sample solution, the method comprising: injecting a sample solution into a container for preparation; subjecting the sample solution to a treatment of agitation by shaking in which the sample solution is agitated by applying a light vibration to the container; and subjecting the sample solution to a treatment of agitation by pipetting in which the sample solution is agitated by pipetting the sample solution in the container.

### TECHNICAL FIELD

The present invention relates to a method for obtaining a sample solution containing nucleic acid from a test sample in order to separate and purify nucleic acid from the test sample containing nucleic acid, and more particularly, to a method for preparing a sample solution in which the pretreatment process is efficiently automated, and a sample solution preparing apparatus.

### BACKGROUND ART

Deoxyribonucleic acid (DNA) is used in a variety of forms, and for example, is commonly used for the detection of pathogenic factors for human and the diagnosis. In general, DNA is available only in very small amounts, and thus the operation of isolation and purification thereof is complicated and time-consuming. Accordingly, various methods for purifying DNA in all forms from all sources with high recovery rate have been developed. For example, the method for purifying DNA disclosed in JP-B-7-51065 below involves the use of a water-soluble organic solvent in purification of DNA, and comprises adsorbing nucleic acid onto a solid phase such as silicon dioxide, silica polymer, magnesium silicate or the like, by using a water-soluble organic solvent such as, for example, ethanol, propanol or isopropanol, and then purifying the nucleic acid by subsequent processes such as washing and desorption, so as to make it possible to purify. DNA with a high recovery rate, and to allow avoiding the use of corrosive as well as toxic compositions such as chaotropes by using a water-soluble organic solvent.

Furthermore, the method for separating and purifying nucleic acid disclosed in Japanese Unexamined Patent Application Publication No. 2003-128691 below is a method for separating and purifying nucleic acid comprising the step of adsorbing nucleic acid onto a solid phase and desorbing the same therefrom, the method enables separation of high purity nucleic acid from a sample solution containing the nucleic acid, by using an organic polymer having hydroxyl groups on the surface as the solid phase, and using a nucleic acid separating and purifying apparatus containing the solid phase in a container having two openings.

### DISCLOSURE OF THE INVENTION

However, the method for purifying DNA as disclosed in JP-B-7-51065 is excellent in the separation performance, but is not satisfactory in convenience, rapidity and automatability, thus having problems that industrial mass production of the adsorption medium of consistent performance is difficult, and that it is difficult to process the adsorption medium into various forms because of its inconvenient handlability.

Meanwhile, the method for separating and purifying nucleic acid as disclosed in Japanese Unexamined Patent Application Publication No. 2003-128691 allows separation of high purity nucleic acid from a sample solution containing the nucleic acid, by using a nucleic acid separating and purifying apparatus containing a solid phase in a container having two openings. However, there still remains a problem of how the sample solution containing nucleic acid should be prepared in the treatment process at a step preceding the process for separating and purifying nucleic acid by extracting the nucleic acid from the sample solution.

During purification of the sample solution, it is necessary to agitate the sample solution; however, for instance, since agitation such as vortexing makes the sample solution to scatter away from the container, there is needed additional time for providing a lid, or the like. However, when the degree of vibration is decreased, a sufficient agitating effect cannot be obtained, and thus the amount of nucleic acid extracted is reduced, thereby lowering the nucleic acid extraction efficiency.

The present invention was achieved upon consideration of such circumstances, and thus it is an object of the invention to provide a method for preparing a sample solution in which pretreatment can be carried out not by agitation by intense shaking but by the combination of mild agitation and pipetting, thus allowing complete automation of the pretreatment process, and a sample solution preparing apparatus. Further, it is another object of the invention to provide a method for separating and purifying nucleic acid by desorbing the nucleic acid by washing or the like, which method is efficient, convenient, fast, excellent in the automation characteristics, and allows to obtain a sample solution containing nucleic acid with reproducibility.

In order to achieve the above-described objects, the following methods are provided.

(1) A method for preparing a sample solution, which is a step preceding a process for separating and purifying a nucleic acid by extracting a nucleic acid from the sample solution, the method comprising:

injecting a sample solution into a container for preparation;

subjecting the sample solution to a treatment of agitation by shaking in which the sample solution is agitated by applying a light vibration to the container; and

subjecting the sample solution to a treatment of agitation by pipetting in which the sample solution is agitated by pipetting the sample solution in the container.

(2) The method for preparing a sample solution as described in (1) above,

wherein the treatment of agitation by shaking involves an agitation by rotatory shaking, and a speed of the rotation is in a range of from 400 to 2000 rpm.

(3) The method for preparing a sample solution as described in (1) or (2) above,

wherein the treatment of agitation by pipetting is carried out in a manner that a volume for one pipetting is in a range of from 50 to 1000 μl.

(4) The method for preparing a sample solution as described in any one of (1) to (3) above,

wherein the treatment of agitation by pipetting is carried out in a manner that a number of a repetition of the pipetting is in a range of from 10 to 100 times.

(5) The method for preparing a sample solution as described in any one of (1) to (4) above, which involves a simultaneous treatment of a plurality of the sample solutions injected into the container.

(6) The method for preparing a sample solution as described in any one of (1) to (5) above,

wherein the process for injecting the sample solution into the container for preparation involves a process of adding a proteolytic enzyme, a sample containing a nucleic acid and a pretreatment solution containing at least one selected from a chaotropic salt, a surfactant, a defoaming agent, a nucleic acid stabilizer and a buffer, and

wherein the proteolytic enzyme, the sample and the pretreatment solution are added in this order,

the pretreatment solution, the sample and the proteolytic enzyme are added in this order, or

the sample, the pretreatment solution and the proteolytic enzyme are added in this order.

(7) The method for preparing a sample solution as described in (6) above,

wherein the process for injecting the sample solution into the container for preparation involves a further addition of a water-soluble organic solvent, after adding the proteolytic enzyme, the sample and the pretreatment solution.

(8) The method for preparing a sample solution as described in (6) or (7) above,

wherein the sample solution is obtained by preparing a whole blood.

(9) The method for preparing a sample solution as described in (7) above,

wherein the water-soluble organic solvent comprises at least one selected from methanol, ethanol, propanol and butanol.

(10) The method for preparing a sample solution as described in (7) above,

wherein the sample solution is contacted with a nucleic acid adsorbing solid phase after the addition of a water-soluble organic solvent.

(11) The method for preparing a sample solution as described in (10) above,

wherein the solid phase is in a membrane form.

(12) The method for preparing a sample solution as described in (10) or (11) above,

wherein the solid phase comprises a silica or a derivative thereof, a diatomaceous earth or an alumina.

(13) The method for preparing a sample solution as described in any one of (10) to (12) above,

wherein the solid phase comprises an organic polymer.

(14) The method for preparing a sample solution as described in (13) above,

wherein the organic polymer is an organic polymer having a polysaccharide structure.

(15) The method for preparing a sample solution as described in (13) or (14) above,

wherein the organic polymer is an acetylcellulose.

(16) The method for preparing a sample solution as described in (13) or (14) above,

wherein the organic polymer is an organic polymer obtained by a saponification of an acetylcellulose or a mixture of acetylcelluloses different from each other in acetyl value.

(17) The method for preparing a sample solution as described in (13) or (14) above,

wherein the organic polymer is a regenerated cellulose.

(18) A sample solution preparing apparatus for preparing a sample solution containing a nucleic acid at a step preceding a process for separating and purifying a nucleic acid by extracting a nucleic acid from the sample solution, the sample solution preparing apparatus comprising:

a container for preparation into which the sample solution is injected;

an agitation by shaking means for agitating the sample solution by applying a light vibration to the container; and

an agitation by pipetting means for agitating the sample solution by pipetting the sample solution in the container.

This method for preparing a sample solution is a method for obtaining a sample solution containing nucleic acid from a test sample, which is carried out prior to the separation and purification of nucleic acid comprising the process of adsorbing the nucleic acid onto a porous membrane and desorbing the same therefrom, wherein a dissolution liquid is added, subsequently agitated first by a shaking operation and then agitated by pipetting, thus a sample solution containing nucleic acid being obtained from the test sample. Accordingly, pretreatment is implemented not by agitation by intense shaking using vortex but by a combination of mild agitation and pipetting, and thereby, it is not necessary to use a lid to prevent scattering, and it is possible to achieve complete automation of the pretreatment process.

Further, this method for preparing a sample solution allows avoiding insufficient agitation, which is likely to occur when the speed of rotation is 400 rpm or less, and also allows avoiding scattering of the sample solution due to the agitation by intense shaking, which is likely to occur when the speed of rotation is 2000 rpm or greater. Thus, mild as well as effective agitation can be performed.

This method for preparing a sample solution also allows avoiding poor agitation due to insufficient amount of pipetting, which is likely to occur when the pipetting volume is 50 μl or less, and also allows avoiding poor agitation due to insufficient mixing of the pipetted sample solution and the non-pipetted sample solution, which is likely to occur when the pipetting volume is 1000 μl or greater.

Furthermore, this method for preparing a sample solution allows avoiding reduction in the yield due to poor agitation by pipetting, which is likely to occur when the number of repetition of pipetting is 10 times or less, and also allows avoiding reduction in the yield due to excessive agitation by pipetting, which is likely to occur when the number of repetition of pipetting is 100 times or greater.

In addition, in this method for preparing a sample solution, it is possible to simultaneously and combinedly prepare and treat the sample solution injected into a plurality of containers, and thus it is possible to carry out a robust operation, without performing the operation of charging containers or injecting sample solution separately by mistake.

In the sample solution preparing apparatus, light vibration is first applied by a means for agitating by shaking to the container for preparation having the sample solution injected in, and then the sample solution in the container is agitated by pipetting by a means for agitating by pipetting. Thus, it is possible to carry out mild as well as effective agitation through agitating in different modes such as light vibration and pipetting. Accordingly, it is not necessary to use vortex which requires a lid, and the lid that is an obstacle to automation is avoidable, thus complete automation of the pretreatment process being achievable.

### BRIEF DESCRIPTION OF THE DRAWING

FIG. 1 is a flowchart indicating the procedure of the method for preparing a sample solution according to the present invention;

FIG. 2 is an external perspective view of the sample solution preparing apparatus according to the invention;

FIG. 3 is a perspective view magnifying the main section of FIG. 2;

FIG. 4 is a diagram illustrating the procedure of the operation of agitation by pipetting in (a), (b) and (c);

FIG. 5 is a time chart indicating the procedure of the agitating operation;

FIG. 6 is a perspective view of the cartridge;

FIG. 7 is a diagram illustrating the procedure of the process of the extraction operation in (a) to (g);

FIG. 8 is a perspective view illustrating the state in which the frontal cover of the nucleic acid extracting apparatus is opened;

FIG. 9 is a diagram outlining the moving head of the nucleic acid extracting apparatus; and

FIG. 10 is a block diagram illustrating the nucleic acid extracting apparatus.

Wherein 25 denotes PIPETTING AGITATING APPARATUS (PIPETTING AGITATING MEANS); 27 denotes VIBRATING APPARATUS (SHAKING AGITATING MEANS); 29 denotes CONTAINER; 30b denotes NUCLEIC ACID-ADSORBING POROUS MEMBRANE (SOLID PHASE); 31 denotes SAMPLE SOLUTION; and 100 denotes SAMPLE SOLUTION PREPARING APPARATUS.

### BEST MODE FOR CARRYING OUT THE INVENTION

Hereinafter, preferred embodiments of the method for preparing a sample solution and the sample solution preparing apparatus according to the present invention will be described with reference to the drawings.

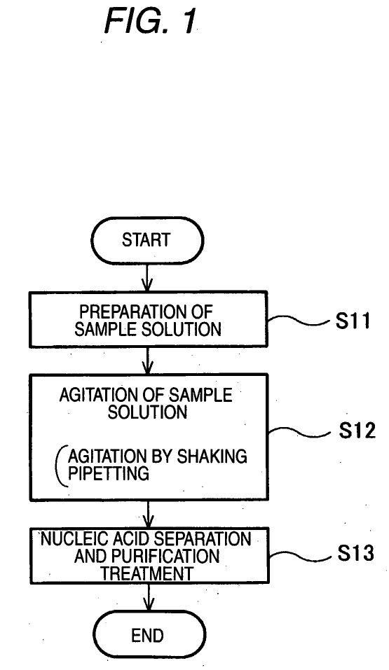

FIG. 1 is a flowchart illustrating the procedure of the method for preparing a sample solution according to the invention.

In the treatment of separation and purification S13 of nucleic acid, the nucleic acid in a sample solution containing nucleic acid is adsorbed onto a nucleic acid-adsorbing solid phase, and then the nucleic acid is desorbed by washing or the like. In such separation and purification of nucleic acid, a sample solution containing nucleic acid is obtained from a test sample prior to the treatment. The sample solution is subjected to separation and purification by agitation, after the preparation S11 of the sample solution. According to the invention, the sample solution of before being subjected to separation and purification is obtained, after addition of the dissolution liquid, first by a shaking operation and then by agitation by pipetting S12.

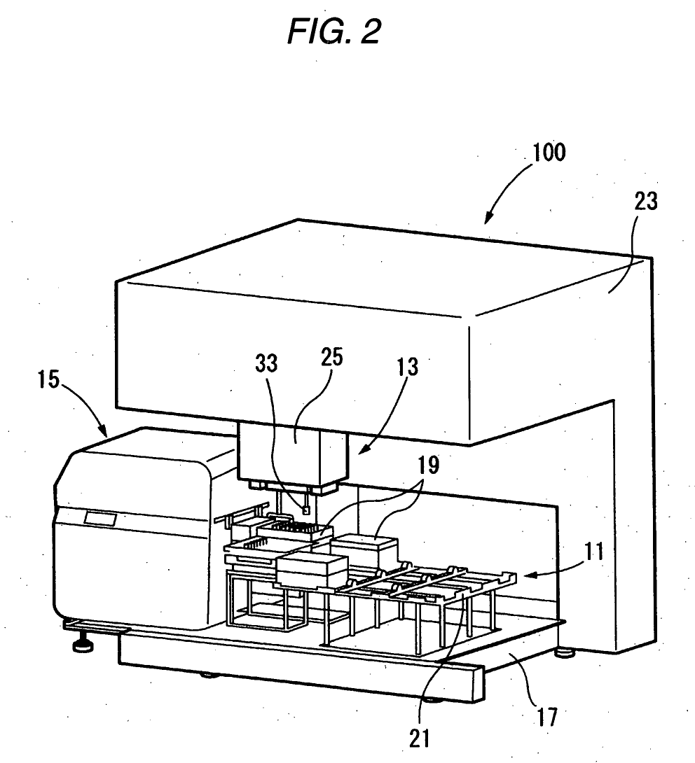

FIG. 2 is an external perspective view of the sample solution preparing apparatus according to the invention.

The sample solution preparing apparatus 100 is installed at a step preceding the nucleic acid extracting apparatus described later. The sample solution preparing apparatus 100 is largely divided into a loader unit 11, an agitating unit 13 and a holding unit 15. The loader unit 11 is installed on a base unit 17. The loader unit 11 is provided with a conveyor 21 having a frame form, and the conveyor 21 allows disposition of a plurality of receiving boxes 19 and conveys these receiving boxes 19 in the XY direction so that the receiving boxes can be supplied to the agitating unit 13. A support unit 23 containing a control unit and the like to be described later is installed at the back of the base unit 17, and the support unit 23 supports a pipetting agitating apparatus 25, which is a means for agitating by pipetting disposed above the agitating unit 13.

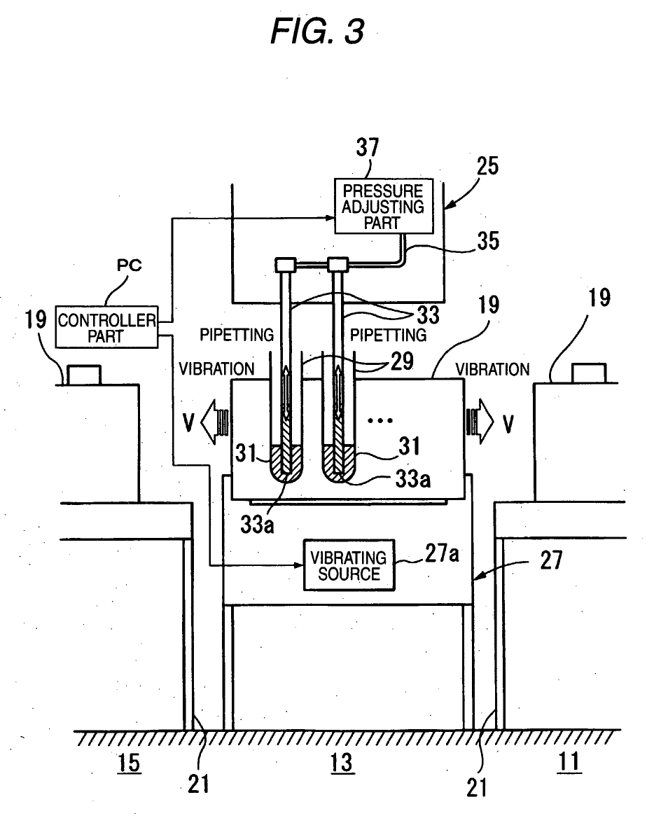

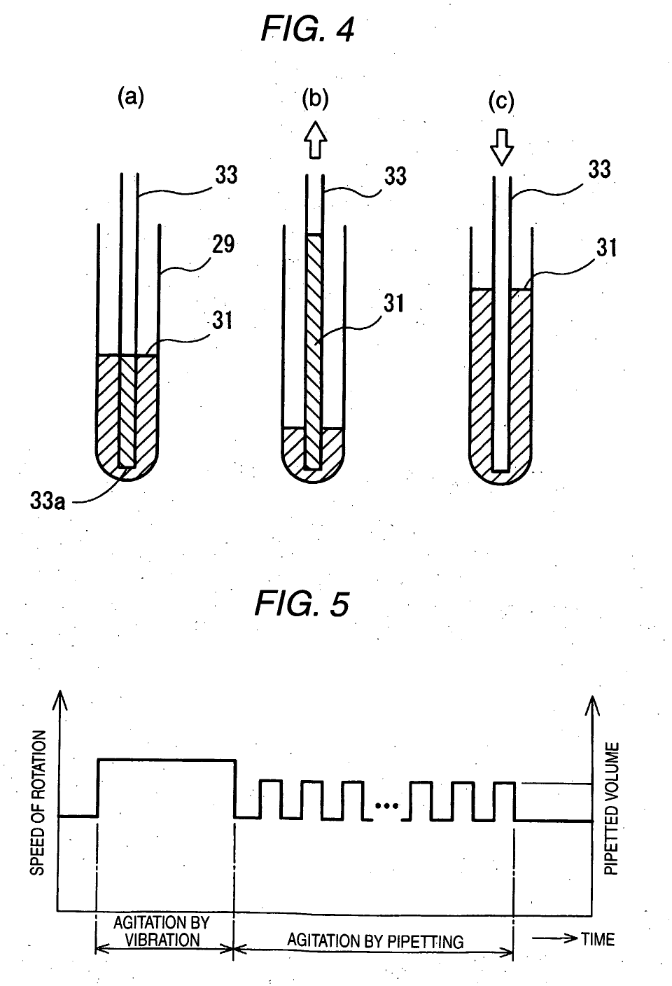

FIG. 3 is a perspective view magnifying the main parts of FIG. 2; FIG. 4 is a diagram illustrating the steps of the operation of agitating by pipetting in (a), (b) and (c); and FIG. 5 is a time chart indicating the step of agitating operation.

At the lower part of the agitating unit 13, a vibrating apparatus 27, which is a means for agitating by shaking, is installed as a part of the conveyor 21. The vibrating apparatus 27 has a vibrating source 27a equipped with an electric motor and the like, in the inside. The operation of the vibrating source 27a is controlled by a PC associated with the control unit comprising a computer and the like. The vibrating apparatus 27 applies light vibration to the receiving boxes 19 that are provided thereabove by controlling the operation of the vibrating source 27a, and enables agitating by shaking of the sample solution 31 in the container 29 which is contained inside the vibrating apparatus.

The treatment of agitation by shaking by the vibrating apparatus 27 is carried out by agitation by rotatory shaking in a single direction (the direction of arrows V in FIG. 3). In the present embodiment, this agitation by rotatory shaking is carried out at a speed of rotation in the range of 400 to 2000 rpm. When the speed of rotation is set in this range, insufficient agitation, which is likely to occur when the speed of rotation is 400 rpm or less, is avoided, while scattering of the sample solution by agitation by intense shaking, which is likely to occur when the speed of rotation is 2000 rpm or greater, is avoided. Thus, mild as well as effective agitation is possible.

The receiving boxes 19 contain a plurality of containers for preparation 29 into which the sample solution is injected. Therefore, the process for preparing the sample solution 31 can simultaneously handle the sample solution 31 injected into a plurality of containers 29. As such, the sample solution 31 injected into a plurality of containers 29 can be subjected to preparation simultaneously and combinedly, thus it being possible to carry out a robust operation without performing the operation of charging the containers 29 or injecting the sample solution separately by mistake.

The receiving boxes 19, being mounted on the vibrating apparatus 27, are disposed right below the pipetting agitating apparatus 25 in the agitating unit 13. The pipetting agitating apparatus 25 has a plurality of pipettes 33 hanging vertically down with respect to the containers 29, and the pipettes 33 have their tip ends 33a inserted into the containers 29 by means of a shifting device that is not shown in the figure. The pipettes 33 are connected to a pressure adjusting unit 37 via a supply line 35, and the pressure adjusting unit 37 is connected to the PC at the control unit by which the pressure adjusting unit is driven and controlled.

In the pipetting agitating apparatus 25, while the tip ends 33a are inserted into the sample solution 31 as shown in FIG. 4(a), the pressure adjusting unit 37 is operated by the PC at the control unit, and the pressure inside the pipettes 33 is lowered so that some of the sample solution 31 is sucked in as shown in FIG. 4(b). Then, the pressure inside the pipettes 33 is increased, and the sample solution 31 is discharged from the pipettes 33 as shown in FIG. 4(c). Accordingly, the sample solution 31 in the containers 29 is agitated by pipetting.

The treatment of agitation by pipetting carried out by the pipetting agitating apparatus 25 is preferably such that the volume of one pipetting is in the range of 50 to 1000 μl. When the volume is set in this range, poor agitation due to insufficient amount of pipetting, which is likely to occur when the pipetting volume is 50 μl or less, is avoided, while poor agitation due to insufficient mixing between the pipetted sample solution and the unpipetted sample solution, which is likely to occur when the pipetting volume is 1000 μl or greater, is avoided.

In addition, the treatment of agitation by pipetting is preferably such that the number of repetition of pipetting is in the range of 10 to 100 times. When the number of repetition is set in this range, reduction in the yield due to poor agitation by pipetting, which is likely to occur when the number of repetition of pipetting is 10 times or less, is avoided, while reduction in the yield due to excessive agitation by pipetting, which is likely to occur when the number of repetition of pipetting is 100 times or greater, is avoided.

As such, the sample solution preparing apparatus 100 is also used for preparing the sample solution 31 containing nucleic acid at a step preceding the process of separating and purifying the nucleic acid by extracting the nucleic acid from the sample solution 31. With respect to the containers for preparation 29 into which the sample solution 31 is injected, first light vibration is applied thereto by the vibrating apparatus 27, and then the sample solution 31 in the containers 29 is agitated by pipetting by means of this pipetting agitating apparatus 25. Therefore, as shown in FIG. 5, it is possible to carry out mild as well as effective agitation by means of agitation in different modes such as light vibration and pipetting. Accordingly, it is not necessary to use vortex which requires a lid, and the lid which is an obstacle to automation is avoidable, thus complete automation of the pretreatment process being achievable.

Here, the process for injecting the sample solution 31 into the containers for preparation 29 can be said to be a process for adding a proteolytic enzyme, a sample containing nucleic acid, and a pretreatment solution containing at least one selected from a chaotropic salt, a surfactant, a defoaming agent, a nucleic acid stabilizer and a buffer, in the described order. Further, the process for injecting the sample solution 31 may be a process for adding the pretreatment solution, the sample and the proteolytic enzyme, in the described order. The process for injecting the sample solution 31 may be also a process for adding the sample, the pretreatment solution and the proteolytic enzyme, in the described order.

The process for injecting the sample solution in the containers for preparation 29 may be a process for further adding a water-soluble organic solvent, after adding the proteolytic enzyme, the sample and the pretreatment solution. In this case, the sample solution 31 can be prepared from the whole blood. Furthermore, the water-soluble organic solvent can contain at least one selected from methanol, ethanol, propanol and butanol.

Next, the nucleic acid extracting apparatus 200 for extracting nucleic acid from the sample solution 31 prepared by the sample solution preparing apparatus 100 will be described.

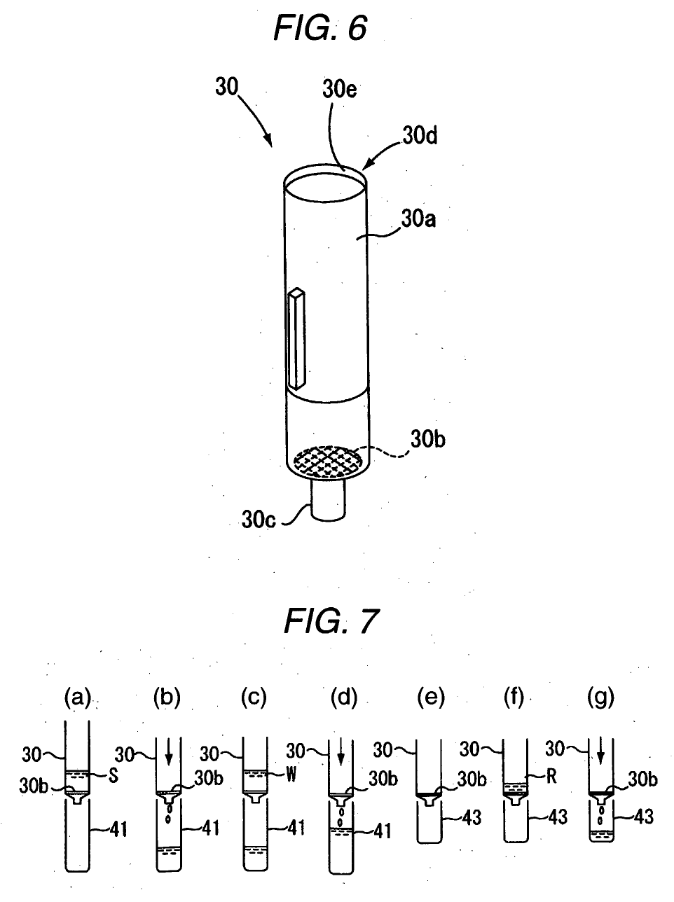

FIG. 6 is a perspective view of a cartridge.

The sample solution 31 prepared by the sample solution preparing apparatus 100 is transferred from the containers 29 to the cartridge 30 of the nucleic acid extracting apparatus 200. The cartridge 30 has a cylindrical main body 30a having the upper end opened and maintaining a nucleic acid-adsorbing porous membrane 30b, which is the solid phase, at the bottom. The part below the nucleic acid-adsorbing porous membrane 30b of the cylindrical main body 30a is shaped into a rod, and a discharging unit 30c having a narrow pipe nozzle shape is protruded from the center of the lower end to a predetermined length. After the sample solution, a washing solution and a recovering solution to be described later are separately injected from the upper opening 30d of the cartridge 30, pressurized air is introduced from the upper opening 30d, and the respective solutions flow down from the discharge unit 30c through the nucleic acid-adsorbing porous membrane 30b into the waste liquor container or recovering container to be described later for discharging. Further, as illustrated in the figure, the cylindrical main body 30a has a structure in which the main body is divided into an upper part and a lower part, which are bonded and adhered. The upper opening 30d has an inclined surface 30e resulting from cutting of the inner peripheral surface into a tapered shape, and this inclined surface 30e is formed to approximately fit the inclined outer peripheral surface at the pressurized nozzle tip end of a pressurized air supply device in the nucleic acid extracting apparatus described below.

FIG. 7 is a process flowchart illustrating the procedure of the nucleic acid extraction as steps (a) to (g).

The process for extracting nucleic acid by means of a nucleic acid extracting apparatus will be described.

The treatment of nucleic acid extraction basically performs extraction of nucleic acid by the extracting process as described in (a) to (g) of FIG. 7. First, in step (a) of FIG. 7, a sample solution S containing solubilized nucleic acid is injected into the cartridge 30 disposed on the waste liquor container 41. In the subsequent step (b), pressurized air is introduced into the cartridge 30 to pressurize the cartridge, the sample solution S is passed through the nucleic acid-adsorbing porous membrane 30b, and the liquid-phase component passed through the nucleic acid-adsorbing porous membrane 30b for the adsorption of the nucleic acid is discharged to the waste liquor container 41.

In the subsequent step (c), the washing solution W is automatically injected into the cartridge 30, and pressurized air is introduced into the cartridge 30 to pressurize the cartridge at step (d). While the nucleic acid is maintained on the nucleic acid-adsorbing porous membrane 30b, other impurities are removed by washing, and the washing solution W passed through the porous membrane is discharged into the waste liquor container 41. These step (c) and step (d) may be repeated a number of times.

Thereafter, the waste liquor container 41 disposed below the cartridge 30 is replaced with the recovering container 43 in step (e), and then the recovering solution R is automatically injected into the cartridge 30 in step (f). In step (g), pressurized air is introduced into the cartridge 30 to pressurize the cartridge, the affinity of the nucleic acid to the nucleic acid-adsorbing porous membrane 30b is attenuated to detach the adsorbed nucleic acid, and the recovering solution R containing the detached nucleic acid is discharged into the recovering container 43 for recovery.

In the cartridge 30, the nucleic acid-adsorbing porous membrane 30b is fundamentally a porous body allowing the nucleic acid to pass through, and its surface has a property of adsorbing the nucleic acid in the sample solution by chemical affinity. The affinity is retained during washing with a washing solution, while the adsorbing power of the nucleic acid is attenuated during recovery with a recovering solution so that the nucleic acid is detached.

Next, the nucleic acid extracting apparatus used in the above-described treatment of extracting nucleic acid will be described.

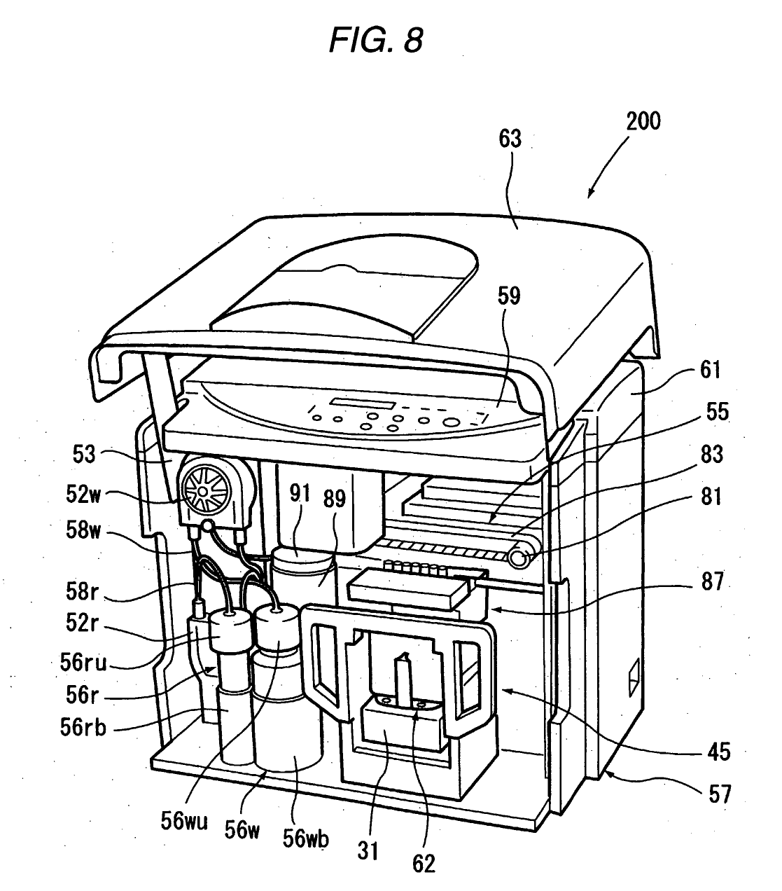

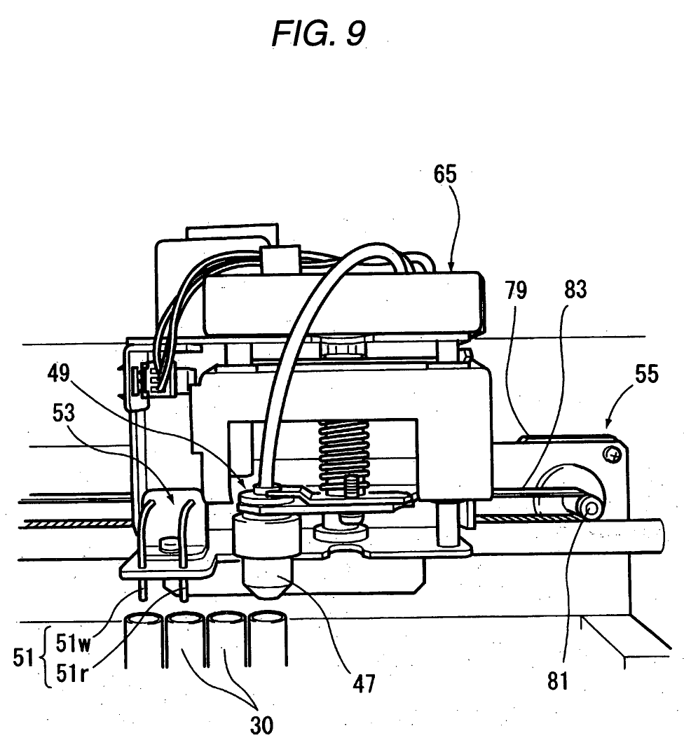

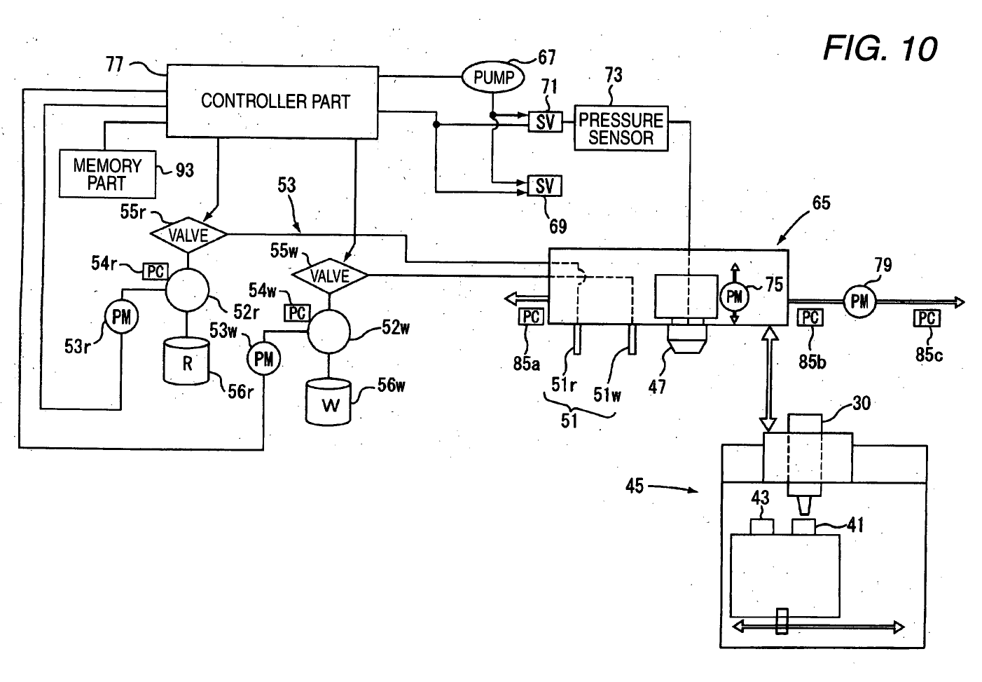

FIG. 8 is a perspective view illustrating the state of the frontal cover of the nucleic acid extracting apparatus being opened; FIG. 9 is a schematic diagram outlining the moving head of the nucleic acid extracting apparatus; and FIG. 10 is a schematic block diagram outlining the nucleic acid extracting apparatus.

The nucleic acid extracting apparatus 200 may include a holding device 45 for arranging and holding a plurality of cartridges 30 containing a filter member in the container, a plurality of waste liquor containers 41 (See FIG. 10) containing the waste liquor, and a plurality of recovering containers 43 (See FIG. 10) containing the recovering solution containing nucleic acid; a pressurized air supply device 49 (See FIG. 9) for introducing pressurized air from a single pressurizing nozzle 47 to the cartridges 30; a separate injection device 53 (See FIG. 9) having a separate injection nozzle 51 which separately injects the washing solution and the recovering solution to the cartridges 30; and a moving device 55 for moving the pressurizing nozzle 47 of the pressurized air supply device 49 and the holding device 45 relatively to each other. The filter member used is a nucleic acid-adsorbing solid phase (a nucleic acid-adsorbing porous membrane as used herein).

The main body of apparatus 57 of the nucleic acid extracting apparatus 200 further includes, in addition to the holding device 45, the pressurized air supply device 49 and the separate injection device 53, a main body unit 61 which is box-shaped with an open frontal side, containing the moving device 55 and the like, and simultaneously providing a control panel 59 on the ceiling; and a frontal cover 63 covering the open side of the main body unit 61.

The pressurized air supply device 49 includes a moving head 65 as a movable body shifting up and down; a single pressurizing nozzle 47 installed on this moving head 65; an air pump 67 generating pressurized air; a relief valve 69; a check valve 71 installed on the side of the pressurizing nozzle 47 to open and close the air supply route; a pressure sensor 73 installed on the side of the pressurizing nozzle 47; and a means for nozzle shifting to shift the pressurizing nozzle 47 up and down. The means for nozzle shifting performs the shifting movement by means of a nozzle shifting motor 75 such as a pulse motor, and a screw-nut device connected thereto. This constitution allows sequential supply of pressurized air to the cartridges 30. The air pump 67, relief valve 69 and pressurizing nozzle 47 respectively operate on the basis of the control commands from the control unit 77.

The moving head 65 includes a head moving motor 79 as a means for movement, installed inside the main body of the apparatus 57, such as a pulse motor; a driving-side pulley 81 driven to rotate by the head moving motor 79; a vertically moving-side pulley (not shown in the figure) freely rotating to adjust tension; and a timing belt 83 bridging between the driving-side pulley 81 and the vertically moving-side pulley. The head moving motor 79 is driven by the feedback control for the detection by the photosensors 85a to 85c, so as to shift the moving head 65 along the direction of the arrangement of the cartridges 30.

The pressurizing nozzle 47 is installed on the moving head 65 to possibly move up and down, with more power exerted on the lower side, and the outer peripheral surface at the lower tip end of the pressurizing nozzle 47 is conically shaped. Thus, when the pressurizing nozzle 47 moves downward, the upper opening 30d of the cartridge disposed on the cartridge holder 87 is contacted with the tip end of the pressurizing nozzle 47, so that the inclined surface 30e of the cartridge 30 cut into a tapered shape adheres to the conical surface of the tip end of the pressurizing nozzle 47 to seal the cartridge 30. In such sealed state, pressurized air can be supplied to the cartridge 30 without leakage.

The relief valve 69 operates by opening to the atmosphere when air is discharged from the channel between the air pump 67 and the check valve 71. The check valve 71 operates by selective opening, and an air circuit is constituted so that pressurized air is introduced from the air pump 67 to the cartridge 30 through the pressurizing nozzle 47. The above constitution allows formation of an air supply channel between the air pump 67 to the cartridge 30.

The separate injection device 53 includes a separate injection nozzle for washing solution 51w and a separate injection nozzle for recovering solution 51r, which are together mounted on the above-described moving head 65 that is movable in the direction of the cartridges 30 lined on the cartridge holder 87; a washing solution feeding pump 52w for supplying the washing solution W contained in the washing solution bottle 56w to the separate injection nozzle for washing solution 51w; a recovering solution feeding pump 52r for supplying the recovering solution R contained in the recovering solution bottle 56r to the separate injection nozzle for recovering solution 51r; a waste liquor container 91 disposed on the waste liquor container holder 89; and the like.

The moving head 65 is driven and controlled to stop sequentially above each cartridge 30 by means of the head moving motor 79, and to stop above the waste liquor container 91 upon returning to the original position, in order to maintain a room above each cartridge 30. When a room is secured above each cartridge 30, workability is greatly improved.

The separate injection nozzle for washing solution 51w and the separate injection nozzle for recovering solution 51r have their tip ends bent downward, and the separate injection nozzle for washing solution 51w is connected to the washing solution feeding pump 52w via a valve 55w, with the washing solution feeding pump 52w being connected to the washing solution bottle 56w. The separate injection nozzle for recovering solution 51r is connected to the recovering solution feeding pump 52r via a valve 55r, with the washing solution feeding pump 52r being connected to the recovering solution bottle 56r. The washing solution bottle 56w and the recovering solution bottle 56r are respectively mounted on the frontal side of the main body of apparatus 57 in order to enhance operability. The washing solution feeding pump 52w and the recovering solution feeding pump 52r consist of tube pumps, and are respectively driven and controlled by the pump motors 53w and 53r (pulse motors) to separately inject predetermined amounts of the washing solution W and the recovering solution R on the basis of the detection of position by the sensors 54w and 54r. These pump motors 53w and 53r, and the valves 55w and 55r operate on the basis of the commands from the control unit 77.

When the washing solution W or recovering solution R are separately injected, the valve 55w or 55r is opened, and the pump motor 53w or 53r is driven to rotate the rotor member of the washing solution feeding pump 52w or the recovering solution feeding pump 52r. Accordingly, the washing solution W or recovering solution R is absorbed by the washing solution feeding pump 52w or the recovering solution feeding pump 52r, and is discharged through the separate injection nozzle for washing solution 51w or the separate injection nozzle for recovering solution 51r via the valve 55w or 55r. Upon this discharge, the separate injection nozzle for washing solution 51w or the separate injection nozzle for recovering solution 51r is moved above the cartridges 30. Thus, a predetermined amount of the washing solution W or recovering solution R is separately injected to the cartridges 30.

The washing solution bottle 56w and the recovering solution bottle 56r respectively consist of a container main body 56wb or 56rb, and a cap 56wu or 56ru. The two caps 56wu and 56ru respectively have a suction tube 58w or 58r in a narrow pipe form, and the lower ends of the suction tubes 58w and 58r are open in the vicinity of the bottoms of the container main body 56wb and 56rb, so that the washing solution W and the recovering solution R are sucked in upon the operation of the washing solution feeding pump 52w and the recovering solution feeding pump 52r, respectively.

The devices 45 through 53 as described above are respectively controlled by the control unit 77 connected to the control panel 59 installed at the upper part of the main body of apparatus 57, in accordance with the input operation at the control panel. That is, the devices are driven and controlled on the basis of the program preliminarily stored in the memory unit 93 connected to the control unit 77. Further, each of the devices 45 through 53 is contained in the main body of apparatus 57, as the main body of apparatus 57 is covered in the front by the frontal cover 63 disposed to be freely removable from the main body of apparatus 57.

Therefore, according to the method for preparing a sample solution of the invention, the method comprises, at a step preceding the process for separating and purifying nucleic acid by extracting the nucleic acid from the sample solution 31, and after injecting the sample solution 31 into the container for preparation 29, the treatment of agitation by shaking in which the sample solution 31 is agitated by applying light vibration to the container, and the treatment of agitation by pipetting in which the sample solution 31 is agitated by pipetting the sample solution 31 in the container 29. Thus, the pretreament can be carried out not by agitation by intense shaking such as vortexing, but by a combination of mild agitation and pipetting, and complete automation of the pretreament process can be achieved. As a result, the method for separating and purifying nucleic acid by adsorbing the nucleic acid in the sample solution 31 containing nucleic acid onto the nucleic acid-adsorbing porous membrane and then desorbing the nucleic acid by washing or the like, is efficient, convenient, fast and excellent in the automatability, and allows to obtain the sample solution 31 containing nucleic acid with reproducibility.

In addition, the sample solution preparing apparatus 100 according to the invention has a container for preparation 29 into which the sample solution 31 is injected, a vibrating apparatus 27 for agitating the sample solution 31 by applying light vibration to this container 29, and a pipetting agitating apparatus 25 for agitating the sample solution 31 by pipetting the sample solution 31 in the container 29. Thus, it is not necessary to use vortex where a lid is required, and complete automation of the pretreatment process can be achieved by avoiding the lid which is an obstacle to automation. As a result, the method for separating and purifying nucleic acid by adsorbing the nucleic acid in the sample solution 31 containing nucleic acid onto the nucleic acid-adsorbing porous membrane and desorbing the nucleic acid by washing or the like, is efficient, convenient, fast and excellent in automatability, and allows to obtain the sample solution 31 containing nucleic acid with reproducibility.

Next, the nucleic acid-adsorbing solid phase 11b (a nucleic acid-adsorbing porous membrane as an example used herein) contained in the cartridge 11 will be described in detail.

The nucleic acid-adsorbing solid phase as used herein may contain silica or a derivative thereof, diatomaceous earth or alumina. The solid phase may also contain an organic polymer. The organic polymer is preferably an organic polymer having a polysaccharide structure. The organic polymer may be also acetylcellulose. The organic polymer may be also an organic polymer obtained by subjecting acetylcellulose or a mixture of acetylcelluloses different from each other in acetyl value, to saponification. The organic polymer may be regenerated cellulose. Detailed description will be given on these.

The nucleic acid-adsorbing solid phase 11b contained in the cartridge 11 is basically porous so that the nucleic acid can pass through, and its surface has a property of adsorbing the nucleic acid in the sample solution by chemical affinity. The affinity is retained during washing with a washing solution, while the adsorbing power of the nucleic acid is attenuated upon recovery with a recovering solution so that the nucleic acid is detached.

The nucleic acid-adsorbing solid phase 11b contained in the cartridge 11 for nucleic acid extraction is a porous solid phase to which the nucleic acid is adsorbed by an interaction substantially not involving the ionic bonding. This implies that the use condition for the porous solid phase is that the porous solid phase is not to be “ionized,” and it is believed that changes in the polarity of the environment causes attraction between the nucleic acid and the porous solid phase. Thus, the porous solid phase has excellent separation performance and good washing efficiency, and allows isolation and purification of the nucleic acid. The nucleic acid-adsorbing porous solid phase is preferably a porous solid phase having hydrophilic groups, and it is believed that changes in the polarity of the environment causes attraction between the nucleic acid and the hydrophilic groups of the porous solid phase.

A hydrophilic group refers to a polar group (group of atoms) capable of interacting with water, and includes all groups (groups of atoms) involved in the adsorption of nucleic acid. The hydrophilic group may be favorably one having interaction with water at a medium intensity (See “Groups having not very strong hydrophilicity” in Section “Hydrophilic Groups”, Encyclopedia Chimica, Kyoritsu Shuppan Co., Ltd.), and examples thereof include a hydroxyl group, a carboxyl group, a cyano group, an oxyethylene group and the like. A preferred one is a hydroxyl group.

Here, the porous solid phase having hydrophilic groups refers to a porous solid phase in which the material forming the porous solid phase itself has hydrophilic groups, or a porous solid phase into which hydrophilic groups are introduced by treating or coating the material forming the porous solid phase. The material forming the porous solid phase may be either organic or inorganic. For example, a porous solid phase in which the material forming the porous solid phase itself is an organic material having hydrophilic groups, a porous solid phase in which hydrophilic groups are introduced by treating a porous solid phase of an organic material having no hydrophilic groups, a porous solid phase in which hydrophilic groups are introduced by coating a porous solid phase of an organic material having no hydrophilic groups with a material having hydrophilic groups, a porous solid phase in which the material forming the porous solid phase itself is an inorganic material having hydrophilic groups, a porous solid phase in which hydrophilic groups are introduced by treating a porous solid phase of an inorganic material having no hydrophilic groups, a porous solid phase in which hydrophilic groups are introduced by coating a porous solid phase of an inorganic material having no hydrophilic groups with a material having hydrophilic groups or the like can be used. From the viewpoint of ease of processing, it is preferable to use an organic material such as an organic polymer, as the material forming the porous solid phase.

A porous solid phase of a material having hydrophilic groups may be a porous solid phase or an organic material having hydroxyl groups. Examples of the porous solid phase of organic material having hydroxyl groups include porous solid phases formed from polyhydroxyethylacrylic acid, polyhydroxyethylmethacrylic acid, polyvinyl alcohol, polyvinylpyrrolidone, polyacrylic acid, polymethacrylic acid, polyoxyethylene, acetylcellulose, a mixture of acetylcelluloses different from each other in acetyl value, or the like. A porous solid phase of an organic material having a polysaccharide structure can be particularly preferably used.

As the porous solid phase of an organic material having hydroxyl groups, a solid phase of an organic polymer consisting of a mixture of acetylcelluloses different from each other in acetyl value can be preferably used. As the mixture of acetylcelluloses different from each other in acetyl value, a mixture of triacetylcellulose and diacetylcellulose, a mixture of triacetylcellulose and monoacetylcellulose, a mixture of triacetylcellulose, diacetylcellulose and monoacetylcellulose, and a mixture of diacetylcellulose and monoacetylcellulose can be preferably used.

In particular, a mixture of triacetylcellulose and diacetylcellulose can be preferably used. The mixing ratio (mass ratio) of triacetylcellulose and diacetylcellulose is preferably 99:1 to 1:99, and more preferably 90:10 to 50:50.

A more preferred organic material having hydroxyl groups may be exemplified by the surface saponification products of acetylcellulose described in JP-A No. 2003-128691. The surface saponification product of acetylcellulose is a product obtained by saponifying a mixture of acetylcelluloses different from each other in acetyl value, and the saponification product of a mixture of triacetylcellulose and diacetylcellulose, the saponification product of a mixture of triacetylcellulose and monoacetylcellulose, the saponification product of a mixture of triacetylcellulose, diacetylcellulose and monoacetylcellulose, and the saponification product of a mixture of diacetylcellulose and monoacetylcellulose can be preferably used. More preferably, the saponification product of a mixture of triacetylcellulose and diacetylcellulose are used. The mixing ratio (mass ratio) of a mixture of triacetylcellulose and diacetylcellulose is preferably 99:1 to 1:99. More preferably, the mixing ratio of a mixture of triacetylcellulose and diacetylcellulose is 90:10 to 50:50. In this case, the degree of saponification treatment (rate of saponification) can be controlled by the amount (density) of the hydroxyl groups on the solid phase surface. In order to increase the separation efficiency of nucleic acid, it is preferable that the amount (density) of hydroxyl groups is large. For example, in the case of acetylcelluloses such as triacetylcellulose, the rate of saponification (rate of surface saponification) is preferably about 5% or greater, and more preferably 10% or greater. Furthermore, in order to increase the surface area of the organic polymer having hydroxyl groups, the porous solid phase of acetylcellulose is preferably subjected to saponification. In this case, the porous solid phase may be a porous membrane having symmetry in the surface and the interior, but a porous membrane having dissymmetry in the surface and the interior can be preferably used.

The treatment of saponification refers to the contacting of acetylcellulose with a solution for saponification treatment (for example, an aqueous solution of sodium hydroxide). Thus, the portion of the acetylcellulose contacted with the solution for saponification treatment is changed to regenerated cellulose, where hydroxyl groups are introduced. The regenerated cellulose thus produced is different from the original cellulose in the crystalline state or the like.

Further, in order to change the rate of saponification, it is preferable to carry out the saponification treatment by changing the concentration of sodium hydroxide. The rate of saponification can be easily measured by NMR, IR or XPS (for example, the rate of saponification can be determined by the degree of peak reduction for a carbonyl group).

As the method for introducing hydrophilic groups to a porous solid phase of an organic material having no hydrophilic groups, graft polymer chains having hydrophilic groups in the polymer chains or in the side chains can be bound to the porous solid phase.

As the method for binding graft polymer chains to a porous solid phase of organic material, mention may be made of two methods including a method for chemically binding graft polymer chains to the porous solid phase, and a method for polymerizing a compound having a polymerizable double bond, with the porous solid phase used as the starting point, to obtain graft polymer chains.

First, in the method for attaching the porous solid phase and the graft polymer chains by chemical binding, the polymer chains can be grafted by using a polymer having a functional group which is reactive with the porous solid phase, at the terminals or in the side chains of the polymer, and chemically reacting this functional group of the polymer with the functional group of the porous solid phase. The functional group which is reactive with the porous solid phase is not particularly limited as long as it can react with the functional group of the porous solid phase, but examples thereof include a silane coupling group such as alkoxysilane, an isocyanate group, an amino group, a hydroxyl group, a carboxyl group, a sulfonic acid group, a phosphoric acid group, an epoxy group, an allyl group, a methacryloyl group, an acryloyl group and the like.

A particularly useful compound as the polymer having a reactive functional group at the terminals or in the side chains of the polymer may be exemplified by a polymer having trialkoxysilyl groups at the polymer terminals, a polymer having amino groups at the polymer terminals, a polymer having carboxyl groups at the polymer terminals, a polymer having epoxy groups at the polymer terminals and a polymer having isocyanate groups at the polymer terminals. The polymer used for this purpose is not particularly limited as long as it has hydrophilic groups that are involved with the adsorption of nucleic acid, but specific examples thereof include polyhydroxyethylacrylic acid and polyhydroxyethylmethacrylic acid and salts thereof, polyvinyl alcohol, polyvinylpyrrolidone, polyacrylic acid and polymethacrylic acid and salts thereof, polyoxyethylene and the like.

The method for forming graft polymer chains by polymerizing a compound having a polymerizable double bond, with the porous solid phase used as the starting point, is generally referred to as surface graft polymerization. The method for surface graft polymerization refers to a method for imparting an active species on the substrate surface by means of plasma irradiation, photoirradiation, heating or the like, and binding a compound having a polymerizable double bond that is disposed to be in contact with the porous solid phase, to the porous solid phase by polymerization.

A compound which is useful for forming graft polymer chains bound to the substrate is required to have two features such as one of having a polymerizable double bond, and the other of having a hydrophilic group that is involved with the adsorption of nucleic acid. For such compound, any compound among polymers, oligomers and monomers having hydrophilic groups can be used, as long as the compound has a double bond in the molecule. A particularly useful compound is a monomer having a hydrophilic group.

Specific examples of the particularly useful monomer having a hydrophilic group include the following monomers. For example, monomers containing hydroxyl-like groups such as 2-hydroxyethylacrylate, 2-hydroxyethylmethacrylate, glycerol monomethacrylate and the like can be particularly preferably used. Carboxyl group-containing monomers such as acrylic acid, methacrylic acid and the like, or alkali metal salts and amine salts thereof also can be preferably used.

As a different method for introducing hydrophilic groups to the porous solid phase of an organic material having no hydrophilic groups, a material having hydrophilic groups can be coated. The material to be used for the coating is not particularly limited as long as the material has hydrophilic groups that are involved with the adsorption of nucleic acid, but from the viewpoint of ease of operation, polymers of organic material are preferred. Examples of the polymers include polyhydroxyethylacrylic acid and polyhydroxyethylmethacrylic acid and salts thereof, polyvinyl alcohol, polyvinylpyrrolidone, polyacrylic acid and polymethacrylic acid and salts thereof, polyoxyethylene, acetylcellulose, mixtures of acetylcelluloses different from each other in acetyl value, and the like, and a polymer having a polysaccharide structure is preferred.

In addition, a porous solid phase of an organic material having no hydrophilic group can be coated with acetylcellulose or a mixture of acetylcelluloses different from each other in acetyl value, and then the coated acetylcellulose or the mixture of acetylcelluloses different from each other in acetyl value can be subjected to saponification. In this case, the rate of saponification is preferably about 5% or greater. Moreover, the rate of saponification is more preferably about 10% or greater.

The porous solid phase which is an inorganic material having hydrophilic groups may be exemplified by porous solid phases containing silica or a derivative thereof, diatomaceous earth or alumina as described above. The porous solid phase containing a silica compound may be exemplified by glass filter. Further, mention may be made of the porous silica thin membrane as described in Japanese Patent No. 3058342. This porous silica thin membrane can be produced by spreading on a substrate, a spreading solution containing a cationic amphiphilic material capable of forming a bimolecular layer, subsequently conditioning the multilayered thin membrane of bimolecular layer of the amphiphilic material by removing the solvent from the liquid membrane on the substrate, contacting the multilayered thin membrane of bimolecular layer with a solution containing a silica compound, and then removing by extraction the multilayered thin membrane of bimolecular layer.

As the method for introducing hydrophilic groups to a porous solid phase of an inorganic material having no hydrophilic group, mention may be made of two methods including a method for chemically binding the porous solid phase with graft polymer chains, and a method for polymerizing graft polymer chains using a monomer having a hydrophilic group which has a double bond in the molecule and, with the porous solid phase used as the starting point.

In the case of attaching the porous solid phase with the graft polymer chains by chemical binding, a functional group which is reactive with the functional group at the terminal of the graft polymer chains is introduced to the inorganic material, and the graft polymer is chemically bound to the inorganic material. In the case of polymerizing graft polymer chains using a monomer having a hydrophilic group which has a double bond in the molecule, with the porous solid phase used as the starting point, a functional group which serves as the starting point for the polymerization of the compound having double bond is introduced into the inorganic material.

As the graft polymer having hydrophilic groups and the monomer having a hydrophilic group which has a double bond in the molecule, those graft polymers having hydrophilic groups and those monomers having a hydrophilic group which have a double bond in the molecule described for the above-described method for chemically binding the porous solid phase of an organic material having no hydrophilic groups and graft polymer chains can be preferably used.

As a different method for introducing hydrophilic groups to the porous solid phase of an inorganic material having no hydrophilic groups, a material having hydrophilic groups can be coated. The material to be used for the coating is not particularly limited as long as the material has hydrophilic groups that are involved with the adsorption of nucleic acid, but from the viewpoint of ease of operation, polymers of organic material are preferred. Examples of the polymers include polyhydroxyethylacrylic acid and polyhydroxyethylmethacrylic acid and salts thereof, polyvinyl alcohol, polyvinylpyrrolidone, polyacrylic acid and polymethacrylic acid and salts thereof, polyoxyethylene, acetylcellulose, mixtures of acetylcelluloses different from each other in acetyl value, and the like, and a polymer having a polysaccharide structure is preferred.

In addition, a porous solid phase of an inorganic material having no hydrophilic groups can be coated with acetylcellulose or a mixture of acetylcelluloses different from each other in acetyl value, and then the coated acetylcellulose or the mixture of acetylcelluloses different from each other in acetyl value can be subjected to saponification. In this case, the rate of saponification is preferably about 5% or greater. Moreover, the rate of saponification is more preferably about 10% or greater.

As the porous solid phase of an inorganic material having no hydrophilic groups, mention may be made of porous solid phases prepared by processing metals such as aluminum, glass, cement, ceramics such as porcelain, or new ceramics, silicon, activated carbon or the like.

The nucleic acid-adsorbing porous solid phase can be used after being formed into a membrane form as described above. In addition to that, the nucleic acid-adsorbing porous solid phase can be also used after being formed into a particulate form or block form in accordance with the shape of the cartridge or the like.

When the nucleic acid-adsorbing porous solid phase is formed into a membrane form, the nucleic acid-adsorbing porous membrane is capable of permitting a solution to pass through the interior, and thus the thickness of the membrane is 10 μm to 500 μm. More preferably, the thickness is 50 μm to 250 μm. In view of the ease of washing, a membrane having a smaller thickness is more desirable.

The nucleic acid-adsorbing porous membrane which is capable of permitting a solution to pass through the interior has a minimum pore size of 0.22 μm or greater. More preferably, the minimum pore size is 0.5 μm or greater. Further, it is desirable to use a porous membrane having a ratio of the maximum pore size and the minimum pore size of two or greater. Then, it is possible to obtain a sufficient surface area for the nucleic acid to adsorb thereon, while it is not easily plugged. Even more preferably, the ratio of the maximum pore size and the minimum pore size is 5 or greater.

The nucleic acid-adsorbing porous membrane which is capable of permitting a solution to pass through the interior has a porosity of 50 to 95%. More preferably, the porosity is 65 to 80%. The bubble point is preferably from 0.1 to 10 kgf/cm². More preferably, the bubble point is from 0.2 to 4 kgf/cm².

The nucleic acid-adsorbing porous membrane which is capable of permitting a solution to pass through the interior preferably has a pressure loss of 0.1 to 100 kPa. Accordingly, uniform pressure can be obtained upon the occurrence of overpressure. More preferably, the pressure loss is from 0.5 to 50 kPa. Here, the pressure loss refers to the minimum pressure required from the membrane to allow water to pass through per 100 μm of the membrane thickness.

For the nucleic acid-adsorbing porous membrane which is capable of permitting a solution to pass through the interior, the amount of water permeated when water is passed through at 25° C. and at a pressure of 1 kg/cm² is preferably 1 to 5000 mL per minute per 1 cm² of the membrane. More preferably, the amount of water permeated when water is passed through at 25° C. and at a pressure of 1 kg/cm² is preferably 5 to 1000 mL per minute per 1 cm² of the membrane.

For the nucleic acid-adsorbing porous membrane which is capable of permitting a solution to pass through the interior, the amount of nucleic acid adsorbed per 1 mg of the porous membrane is preferably 0.1 μg or greater. More preferably, the amount of nucleic acid adsorbed per 1 mg of the porous membrane is 0.9 μg or greater.

The nucleic acid-adsorbing porous membrane which is capable of permitting a solution to pass through the interior is preferably a cellulose derivative which does not dissolve within 1 hour but dissolves within 48 hours, when a square-shaped porous membrane with its each side being 5 mm is immersed in 5 mL of trifluoroacetic acid. Further, more preferred is a cellulose derivative which dissolves within 1 hour when a square-shaped porous membrane with its each side being 5 mm is immersed in 5 mL of trifluoroacetic acid, but which does not dissolve within 24 hours when the same specimen is immersed in 5 mL of dichloromethane.

When a sample solution containing nucleic acid is passed through the nucleic acid-adsorbing porous membrane, it is preferable to pass the sample solution from one side to the other side, from the perspective that the membrane can contact the sample solution with the porous membrane uniformly. When a sample solution containing nucleic acid is passed through the nucleic acid-adsorbing porous membrane, it is preferable to pass through the sample solution from the side having a larger pore size of the porous membrane to the side having a smaller pore size, from the perspective that the membrane is not easily plugged.

When a sample solution containing nucleic acid is passed through the nucleic acid-adsorbing porous membrane, the flow rate is preferably from 2 to 1500 μL/sec per cm² of the surface area of the membrane, in order to provide an appropriate contact time for the solution with the porous membrane. When the contact time for the solution with the porous membrane is excessively short, a sufficient effect of nucleic acid extraction cannot be obtained. When the contact time is excessively long, it is not desirable from the viewpoint of operability. Furthermore, the flow rate is preferably from 5 to 700 μL/sec per cm² of the surface area of the membrane.

A single nucleic acid-adsorbing porous membrane which is capable of permitting the solution used to pass through the interior may be used, but a plurality of such membranes may be also used. The plural nucleic acid-adsorbing porous membranes may be identical or different.

The plural nucleic acid-adsorbing porous membrane may be composed of a combination of nucleic acid-adsorbing porous membranes of an inorganic material and nucleic acid-adsorbing porous membranes of an organic material. For example, mention may be made of a combination of a glass filter and a porous membrane of regenerated cellulose. Further, the plural nucleic acid-adsorbing porous membranes may be composed of a combination of nucleic acid-adsorbing porous membranes of an inorganic material and non-nucleic acid-adsorbing porous membranes of an organic material, and may be exemplified by a combination of a glass filter and a porous membrane of nylon or polysulfone.

Next, the sample solution will be described in detail.

<Sample Solution Containing Nucleic Acid>

The sample solution containing nucleic acid can be obtained by treating a pretreatment solution containing at least one selected from a nucleic acid stabilizer, a chaotropic salt, a surfactant, a buffer, a defoaming agent and a proteolytic enzyme, with a nucleic acid solubilizing reagent, and particularly preferably the solution is a solution obtained by adding a water-soluble organic solvent.

(Test Sample)

The test sample that can be used in the invention is not particularly limited as long as the test sample contains nucleic acid. For example, mention may be made of body fluids such as collected whole blood, blood plasma, blood serum, urine, faeces, semen, saliva or the like in the field of diagnosis, or biological materials such as plants (or parts thereof), animals (or parts thereof), bacteria, viruses or the like. The test sample may be used as received, or a dissolution liquid or homogenate thereof may be also used as the sample.

The “sample” means any sample containing nucleic acid. More specifically, mention may be made of those described with respect to the above-described test samples. There may be one type of the nucleic acid, or two or more types of the nucleic acid in the sample solution. The length of each nucleic acid that is provided to the above-described method for separating and purifying nucleic acid is not particularly limited, and for example, a nucleic acid of any length from a few bps to a few Mbps. In general, from the viewpoint of handlability, the length of the nucleic acid is preferably in the range of a few bps to a few hundred kbps.

According to the invention, the “nucleic acid” may be any DNA or RNA of a single strand or double strand, and the molecular weight thereof is also not limited.

The test sample can be preferably obtained as a sample solution containing nucleic acid by solubilizing the cell membrane, nuclear membrane and the like, and dispersing the nucleic acid in an aqueous solution.

(Defoaming Agent)

The defoaming agent may be exemplified by silicone-based defoaming agents (e.g., silicone oils, dimethylpolysiloxanes, silicon emulsions, modified polysiloxanes, silicon compounds, etc.), alcohol-based defoaming agents (e.g., acetylene glycol, heptanol, ethylhexanol, high alcohols, polyoxyalkylene glycols, etc.), ether-based defoaming agents (e.g., heptylcellosolve, nonylcellosolve-3-heptylcorbitol, etc.), fat- and oil-based defoaming agents (e.g., animal and plant oils, etc.), fatty acid-based defoaming agents (e.g., stearic acid, oleic acid, palmitic acid, etc.), metal soap-based defoaming agents (e.g., aluminum stearate, calcium stearate, etc.), fatty acid ester-based defoaming agents (e.g., natural waxes, tributyl phosphate, etc.), phosphoric acid ester-based defoaming agents (e.g., sodium octylphosphate, etc.), amine-based defoaming agents (e.g., diamylamine, etc.), amide-based defoaming agents (e.g., stearic acid amide, etc.), other defoaming agents (e.g., iron(III) sulfate, bauxite, etc.), or the like. Preferred are silicone-based defoaming agents and alcohol-based defoaming agents. These defoaming agents may be used individually or in combination. It is particularly preferable to use two components in combination, including one from silicone-based defoaming agents and the other from alcohol-based defoaming agents, as the defoaming agent. For the alcohol-based defoaming agent, acetylene glycol-based surfactants are preferred.

The defoaming agent may be added directly to the test sample, or may be contained in the pretreatment solution that is used as the nucleic acid solubilizing reagent. When the defoaming agent is not contained in the pretreatment solution, the timing for the addition of defoaming agent may be before or after the use of the pretreatment solution.

The concentration of the defoaming agent in the sample solution containing nucleic acid is preferably from 0.1 to 10% by mass. (In this specification, % by mass is equal to % by weight.)

(Nucleic Acid Stabilizer)

For the nucleic acid stabilizer, mention may be made of those having an action of deactivating the activity of nucleases. Depending on the test sample, a nuclease or the like degrading the nucleic acid may be originally contained in the test sample, and when the nucleic acid is homogenized, this nuclease may act on the nucleic acid, resulting in a significantly reduced yield. The nucleic acid stabilizer is desirable since it can help the nucleic acid in the test sample to exist stably.

For the nucleic acid stabilizer having an effect of deactivating the activity of nuclease, those compounds generally used as reducing agent can be used. Examples of the reducing agent include hydride compounds such as hydrogen, hydrogen iodide, hydrogen sulfide, lithium aluminum hydride, sodium borohydride, and the like; alkali metals; metals having large electropositivity such as magnesium, calcium, aluminum, zinc and the like, or amalgams thereof; aldehydes; sugars; organic oxides such as formic acid, oxalic acid and the like; mercapto compounds; and the like. Among these, mercapto compounds are preferred. The mercapto compound may be exemplified by N-acetylcystein, mercaptoethanol, alkylmercaptan or the like. In particular, β-mercaptoethanol is preferred. Mercapto compounds may be used individually or in combination of a plurality.

The nucleic acid stabilizer can be used at a concentration of preferably 0.1 to 20% by mass, more preferably 0.3 to 15% by mass, in the pretreatment solution. The mercapto compound can be used at a concentration of preferably 0.1 to 10% by mass, more preferably 0.5 to 5% by mass, in the pretreatment solution.

In addition, as the nucleic acid stabilizer having an effect of deactivating the activity of nuclease, chelating agents can be used. Examples of the chelating agent include ethylenediamine tetraacetate (EDTA), nitrilotriacetate (NTA), EGTA and the like. The chelating agents may be used individually or in combination of a plurality. The chelating agent can be used in the pretreatment solution at a concentration of preferably 1 mmol/L to 1 mol/L, more preferably 5 mmol/L to 100 mmol/L.

(Chaotropic Salt)

For the chaotropic salt, guanidine salts, sodium isocyanate, sodium iodide, potassium iodide or the like can be used. Among these, guanidine salts are preferred. Examples of the guanidine salt include guanidine hydrochloride, guanidine isothiocyanate, and guanidine thiocyanate, and among these, guanidine hydrochloride is preferred. These salts may be used individually or in combination of a plurality. The concentration of the chaotropic salt in the pretreatment solution is preferably 0.5 mol/L or greater, more preferably 0.5 mol/L to 4 mol/L, and even more preferably 1 mol/L to 3 mol/L.

Urea also can be used as the chaotropic substance instead of chaotropic salts.

(Surfactant)

For the surfactant, mention may be made of nonionic surfactants, cationic surfactants, anionic surfactants, and zwitterionic surfactants.

According to the invention, nonionic surfactants and cationic surfactants can be favorably used.

Examples of the nonionic surfactant include polyoxyethylene alkylphenyl ether surfactants, polyoxyethylene alkyl ether surfactants, and fatty acid alkanolamides, and preferably polyoxyethylene alkyl ether surfactants. Among the polyoxyethylene alkyl ether surfactants, POE decyl ether, POE lauryl ether, POE tridecyl ether, POE alkylene decyl ether, POE sorbitan monolaurate, POE sorbitan monooleate, POE sorbitan monostearate, tetraoleic acid polyoxyethylene sorbite, POE alkylamine, and POE acetylene glycol are more preferred.

Examples of the cationic surfactant include cetyltrimethylammonium bromide, dodecyltrimethylammonium chloride, tetradecyltrimethylammonium chloride and cetylpyridinium chloride.

These surfactants may be used individually or in combination of a plurality. The concentration of the surfactant in the pretreatment solution is preferably 0.1 to 20% by mass.

(Buffer)

For the buffer, mention may be made of the conventionally used pH buffers. Preferably, mention may be made of the pH buffers that are conventionally used in biochemical tests. Examples of such buffer include buffers comprising citrate, phosphate or acetate, Tris-HCl, TE (Tris-HCl/EDTA), TBE (Tris-Borate/EDTA), TAE (Tris-Acetate/EDTA), and Good's buffers. Examples of the Good's buffer include MES (2-morpholinoethanesulfonic acid), Bis-Tris (bis(2-hydroxyethyl)iminotris(hydroxylmethyl)methane), HEPES (2-[4-(2-hydroxyethyl)-1-piperazinyl]ethanesulfonic acid), PIPES (piperazine-1,4-bis(2-ethanesulfonic acid)), ACES (N-(2-acetamino)-2-aminoethanesulfonic acid), CAPS(N-cyclohexyl-3-aminopropanesulfonic acid), and TES (N-tris(hydroxymethyl)methyl-2-aminoethanesulfonic acid).

These buffers are preferably contained in the pretreatment solution at a concentration of 1 to 300 mmol/L.

(Proteolytic Enzyme)

For the proteolytic enzyme, mention may be made of serine proteases, cystein proteases, and metal proteases, and at least one proteolytic enzyme can be favorably used. Further, a mixture of a plurality of proteolytic enzymes also can be favorably used.

The pretreatment solution preferably contains proteolytic enzymes from the viewpoints of enhancement in the recovery amount of nucleic acid and the recovery efficiency, reduction of the required amount of the test sample containing nucleic acid, and rapid processing.

The serine protease is not particularly limited, and for example, protease K or the like can be favorably used. The cystein protease is not particularly limited, and for example, papain, cathepsin or the like can be favorably used. The metal protease is not particularly limited, and for example, carboxypeptidase or the like can be favorably used.

The concentration of the proteolytic enzyme in the pretreatment solution is preferably 0.001 IU to 10 IU, more preferably 0.01 IU to 1 IU, per milliliter of the total volume upon addition.

In addition, those proteolytic enzymes not containing nucleases can be favorably used. Further, proteolytic enzymes containing stabilizers also can be favorably used. For the stabilizer, metal ions can be favorably used. Specifically, magnesium ion is preferred, and the magnesium ion can be added in the form of, for example, magnesium chloride or the like. When the proteolytic enzyme contains a stabilizer, it is possible to reduce the amount of the proteolytic enzyme required in the recovery of nucleic acid, and thus to reduce the costs-required for the recovery of nucleic acid.

The concentration of the stabilizer for the proteolytic enzyme in the pretreatment solution is, preferably 1 to 1000 mmol/L, and more preferably 10 to 100 mmol/L.

In the case of using proteases, incubation may be needed. In this case, the conditions for incubation may be such that the environmental temperature is from room temperature to 80° C., and preferably from 40° C. to 70° C.

The proteolytic enzyme may be supplied for the recovery of nucleic acid after being preliminarily mixed with the chaotropic salt, surfactants, buffers and other reagents, as a pretreatment solution (hereinafter, referred to as pretreatment solution A).

Furthermore, the proteolytic enzyme may be supplied as two or more reagents, separately from a pretreatment solution containing the chaotropic salt, surfactants, buffers and other reagents (hereinafter, referred to as pretreatment solution B). In the latter case, the reagent containing the proteolytic enzyme is first mixed with the test sample and then mixed with the pretreatment solution B. It is also possible to first mix the pretreatment solution B with the test sample and then with the proteolytic enzyme.

It is also possible to add dropwise the proteolytic enzyme from the proteolytic enzyme storing container directly in the form of eye drops, to the test sample or the liquid mixture of the test sample and the pretreatment solution B. In this case, operation becomes more convenient.

The pretreatment solution is also preferably supplied in a dried state, that is, as a pretreatment agent. It is also possible to use a container preliminarily containing the proteolytic enzyme in a dried state such as the freeze-dried state. A sample solution containing nucleic acid can be obtained by using the above-mentioned container preliminarily containing the pretreatment agent and/or the proteolytic enzyme in a dried state.

When a sample solution containing nucleic acid is to be obtained by the method described above, the storage stability of the pretreatment agent and the proteolytic enzyme in a dried state is good, and thus the operation can be performed conveniently without affecting the nucleic acid yield.

Moreover, from the viewpoint of enhancing the solubility of the compounds contained in the sample solution, a water-soluble organic solvent may be added to the pretreatment solution. The water-soluble organic solvent may be exemplified by alcohols, acetone, acetonitrile, dimethylformamide or the like. Among these, alcohols are preferred. For the alcohols, primary alcohol, secondary alcohol and tertiary alcohol are all favorable. Specifically, mention may be made of methanol, ethanol, propanol and isomers thereof, butanol and isomers thereof, and the like, and among these, ethanol is particularly preferred. These water-soluble organic solvents may be used individually or in combination of a plurality. The concentration of the water-soluble organic solvent is preferably adjusted to 1 to 20% by mass in the sample solution containing nucleic acid.

{Process of Adsorption to Water-Soluble Organic Solvent}

The sample solution containing nucleic acid is preferably a solution obtained by further adding a water-soluble organic solvent in order to effectively adsorb the nucleic acid in the sample solution to a solid phase, by adding the water-soluble organic solvent to the solution having the nucleic acid solubilized and dispersed, and contacting the solution with the solid phase. That is, it is preferable to obtain a sample solution containing nucleic acid by further adding a water-soluble organic solvent to a solution obtained by treating with the above-described pretreatment solution. Moreover, it is preferable to have a salt present in the obtained sample solution containing nucleic acid, since the salt facilitates the adsorption of the solubilized nucleic acid to the solid phase more efficiently.

The presence of the water-soluble organic solvent and the salt causes destruction of the hydrate structure formed by the water molecules existing around the nucleic acid, and thus solubilization of the nucleic acid into an unstable state. It can be conceived that when the nucleic acid in this state is contacted with the solid phase, there occurs an interaction between the polar groups on the nucleic acid surface and the polar groups on the solid phase surface, and the nucleic acid adsorbs on the surface of the solid phase. In particular, when an organic polymer having hydroxyl groups on the surface is used as the solid phase, it is preferable because of the remarkable adsorption resulting therefrom. According to the method for the invention, it is preferable to mix the mix solution containing solubilized nucleic acid as described above with a water-soluble organic solvent, and to allow a salt to be present in the resulting nucleic acid mix solution, in view of making the nucleic acid unstable.

This water-soluble organic solvent may be exemplified by alcohols, acetone, acetonitrile, dimethylformamide or the like. Among these, alcohols are preferred. For the alcohols, primary alcohol, secondary alcohol and tertiary alcohol are all favorable. Among these, methanol, ethanol, propanol and isomers thereof, and butanol and isomers thereof can be preferably used. More preferably, ethanol can be used. These water-soluble organic solvents may be used individually or in combination of a plurality.

The final concentration of the water-soluble organic solvent in the sample solution containing nucleic acid is preferably 5 to 90% by mass. The concentration of added ethanol within this range does not cause formation of aggregates, and it is particularly preferable to increase the concentration of ethanol as much as possible. The concentration is more preferably 20% by mass to 70% by mass.

The salt whose presence in the obtained nucleic acid mix solution is favored may be exemplified by various chaotropic substances (guanidium salts, sodium iodide, sodium perchlorate), sodium chloride, potassium chloride, ammonium chloride, sodium bromide, potassium bromide, calcium bromide, ammonium bromide or the like. Particularly, guanidium salts are particularly preferred since they have both the effect of dissolving cellular membrane and the effect of solubilizing nucleic acid.

The pH of the obtained sample solution to be used is preferably pH 3 to 10, more preferably pH 4 to 9, and even more preferably pH 5 to 8.

Furthermore, the obtained sample solution containing nucleic acid has a surface tension of preferably 0.05 J/m² or less, a viscosity of preferably 1 to 10,000 mPa, and a specific density preferably in the range of 0.8 to 1.2. When a solution having the properties in these ranges is used in the adsorption step, the solution remaining after the adsorption of the nucleic acid by contacting the sample solution containing nucleic acid with the solid phase, can be easily removed in the washing step.

### FIRST EMBODIMENT

(1) Preparation of Container for Nucleic Acid Separation and Purification

A container for nucleic acid purification with an inner diameter of 7 mm, containing a solid phase for nucleic acid adsorption and having two openings, was prepared from polypropylene.

(2) Nucleic Acid Separating and Purifying Apparatus

A porous membrane obtained by subjecting a triacetylcellulose porous membrane to saponification was used as the nucleic acid-adsorbing porous membrane, and was placed in the nucleic acid-adsorbing porous membrane holder part in the cartridge for nucleic acid purification prepared in (1) above.

(3) Preparation of DNA Solubilizing Reagent and Washing Solution

The DNA solubilizing reagent and washing solution as prescribed in Table 1 were prepared.

TABLE 1

| | |
|---|---|
| DNA | 382 g of Guanidine hydrochloride (Life |
| solubilizing | Technologies Co., Ltd.) |
| reagent | 12.1 g of Tris (Life Technologies Co., Ltd.) |
| | 10 g of Tween 20 (Wako Pure Chemical |
| | Industries, Ltd.) |
| | 1000 ml of Distilled water |
| Washing | 10 mM Tris-HCl 50% ethanol |
| solution | |

(4) Nucleic Acid Purification Operation

200 μl of human whole blood was collected using a vacuum blood collection tube. To this, 200 μl of the DNA solubilizing reagent as prescribed in Table 1 and 20 μl of protease K were added, and the mixture was incubated at 60° C. for 10 minutes. After incubation, 200 μl of ethanol was added and agitated.

Agitation was carried out under the conditions described in Table 2. The pipetting was carried out under the conditions described in Table 3.

After the agitation, the whole blood sample treated as described above was injected into a first opening of the nucleic acid purifying apparatus having a porous membrane or an organic polymer composed of the mixture of acetylcelluloses different from each other in acetyl value prepared in (1) and (2) above, and subsequently the first opening was connected to a pressure difference generating apparatus to pressurize the nucleic acid separating and purifying apparatus. The sample solution containing the injected whole blood sample was passed through the porous membrane to be brought into contact with the porous membrane, and was discharged from the other opening of the nucleic acid separating and purifying apparatus.

Subsequently, the washing solution was injected into the first opening of the nucleic acid separating and purifying apparatus, and the first opening was connected to a pressure difference generating apparatus to pressurize the nucleic acid separating and purifying apparatus. The injected washing solution was passed through the porous membrane and discharged through the other opening. Subsequently, the recovering solution was injected into the first opening of the nucleic acid separating and purifying apparatus, and the first opening was connected to a pressure difference generating apparatus to pressurize the nucleic acid separating and purifying apparatus. The injected recovering solution was passed through the porous membrane and discharged through the other opening.

(5) Conformation of DNA Separation and Purification

An absorption spectrum of the recovering solution at 260 nm was measured to determine the yield of DNA.

The relationship of the agitating conditions and the DNA yield in the Comparative Examples and the Examples is indicated in Table 2, and the same relationship of pipetting conditions is indicated in Table 3.

TABLE 2
Agitating conditions

| | Comparative Example | | | | | Example | | | | | |
|---|---|---|---|---|---|---|---|---|---|---|---|
| | 1 | 2 | 3 | 4 | 5 | 1 | 2 | 3 | 4 | 5 | 6 |
| Shaking | None | 1200 | None | 1200 | — | 1200 | 1600 | 2000 | 1000 | 800 | 400 |
| conditions | | | | | | | | | | | |
| (rpm) | | | | | | | | | | | |
| Shaking | — | 20 sec | — | 1 min | — | 20 sec | 20 sec | 20 sec | 20 sec | 20 sec | 20 sec |
| time | | | | | | | | | | | |
| Number of | None | None | 10x | None | 50x | 10x | 10x | 10x | 10x | 10x | 10x |
| pipetting | | | | | | | | | | | |
| Yield (μg) | 0.3 | 1.2 | 0.9 | 1.2 | 1.5 | 4.3 | 4.2 | 4.5 | 4.1 | 4.2 | 3.9 |

TABLE 3
Pipetting conditions

| | Comparative Example | | | | Example | | |
|---|---|---|---|---|---|---|---|
| | 1 | 2 | 3 | 4 | 1 | 2 | 3 |
| Number of | 5 | 300 | 20 | 100 | 10 | 100 | 20 |
| pipetting | | | | | | | |
| (times) | | | | | | | |
| Volume | 100 | 100 | 10 | 2000 | 100 | 100 | 50 |
| (μl) | | | | | | | |
| Yield | 2.4 | 2.2 | 1.1 | 2.3 | 4.1 | 4.3 | 4.3 |
| (μg) | | | | | | | |

As can be seen from the results in Table 2 and Table 3, a combination of two agitating conditions such as agitation by shaking and agitation by pipetting allowed a remarkable enhancement in the yield of DNA.

### INDUSTRIAL APPLICABILITY

The method for preparing a sample solution according to the present invention includes, after injecting the sample solution into a container for preparation at a step preceding the process for separating and purifying nucleic acid by extracting the nucleic acid from the sample solution, a treatment of agitation by shaking in which the sample solution is agitated by applying light vibration to the container and a treatment of agitation by pipetting in which the sample solution is agitated by pipetting the sample solution in the container. Therefore, the pretreatment can be carried out not by agitation with intense shaking such as vortexing, but by a combination of mild agitation and pipetting, and thus complete automation of the pretreatment process can be achieved. As a result, the method for separating and purifying nucleic acid by adsorbing the nucleic acid in a sample solution containing nucleic acid onto a nucleic acid-adsorbing porous membrane and then desorbing the nucleic acid by washing or the like, is efficient, convenient, fast and excellent in the automation, and allows to obtain a sample solution containing nucleic acid with reproducibility.

The sample solution preparing apparatus according to the invention is a sample solution preparing apparatus for preparing a sample solution containing nucleic acid that has a container for preparation into which the sample solution is injected, a means for agitating by shaking which agitates the sample solution by applying light vibration to the container, and a means for agitating by pipetting which agitates the sample solution by pipetting the sample solution in the container. Accordingly, it is not necessary to use vortex which requires a lid, and the lid which is an obstacle to automation is avoidable, thus complete automation of the pretreatment process being achievable. As a result, the method for separating and purifying nucleic acid by adsorbing the nucleic acid in a sample solution containing nucleic acid onto a nucleic acid-adsorbing porous membrane and then desorbing by washing or the like, is efficient, convenient, fast and excellent in the automation, and a sample solution containing nucleic acid can be obtained with reproducibility.

The entire disclosure of each and every foreign patent application from which the benefit of foreign priority has been claimed in the present application is incorporated herein by reference, as if fully set forth.

1. A method for preparing a sample solution, which is a step preceding a process for separating and purifying a nucleic acid by extracting a nucleic acid from the sample solution, the method comprising:
injecting a sample solution into a container for preparation;
subjecting the sample solution to a treatment of agitation by shaking in which the sample solution is agitated by applying a light vibration to the container; and
subjecting the sample solution to a treatment of agitation by pipetting in which the sample solution is agitated by pipetting the sample solution in the container.

2. The method for preparing a sample solution according to claim 1,
wherein the treatment of agitation by shaking involves an agitation by rotatory shaking, and a speed of the rotation is in a range of from 400 to 2000 rpm.

3. The method for preparing a sample solution according to claim 1,
wherein the treatment of agitation by pipetting is carried out in a manner that a volume for one pipetting is in a range of from 50 to 1000 μl.

4. The method for preparing a sample solution according to claim 1,
wherein the treatment of agitation by pipetting is carried out in a manner that a number of a repetition of the pipetting is in a range of from 10 to 100 times.

5. The method for preparing a sample solution according to claim 1, which involves a simultaneous treatment of a plurality of the sample solutions injected into the container.

6. The method for preparing a sample solution according to claim 1,
wherein the process for injecting the sample solution into the container for preparation involves a process of adding a proteolytic enzyme, a sample containing a nucleic acid and a pretreatment solution containing at least one selected from a chaotropic salt, a surfactant, a defoaming agent, a nucleic acid stabilizer and a buffer, and
wherein the proteolytic enzyme, the sample and the pretreatment solution are added in this order,
the pretreatment solution, the sample and the proteolytic enzyme are added in this order, or
the sample, the pretreatment solution and the proteolytic enzyme are added in this order.

7. The method for preparing a sample solution according to claim 6,
wherein the process for injecting the sample solution into the container for preparation involves a further addition of a water-soluble organic solvent, after adding the proteolytic enzyme, the sample and the pretreatment solution.

8. The method for preparing a sample solution according to claim 6,
wherein the sample solution is obtained by preparing a whole blood.

9. The method for preparing a sample solution according to claim 7,
wherein the water-soluble organic solvent comprises at least one selected from methanol, ethanol, propanol and butanol.

10. The method for preparing a sample solution according to claim 7,
wherein the sample solution is contacted with a nucleic acid adsorbing solid phase after the addition of a water-soluble organic solvent.

11. The method for preparing a sample solution according to claim 10,
wherein the solid phase is in a membrane form.

12. The method for preparing a sample solution according to claim 10,
wherein the solid phase comprises a silica or a derivative thereof, a diatomaceous earth or an alumina.

13. The method for preparing a sample solution according to claim 10,
wherein the solid phase comprises an organic polymer.

14. The method for preparing a sample solution according to claim 13,
wherein the organic polymer is an organic polymer having a polysaccharide structure.

15. The method for preparing a sample solution according to claim 13,
wherein the organic polymer is an acetylcellulose.

16. The method for preparing a sample solution according to claim 13,
wherein the organic polymer is an organic polymer obtained by a saponification of an acetylcellulose or a mixture of acetylcelluloses different from each other in acetyl value.

17. The method for preparing a sample solution according to claim 13,
wherein the organic polymer is a regenerated cellulose.

18. A sample solution preparing apparatus for preparing a sample solution containing a nucleic acid at a step preceding a process for separating and purifying a nucleic acid by extracting a nucleic acid from the sample solution, the sample solution preparing apparatus comprising:
a container for preparation into which the sample solution is injected;
an agitation by shaking means for agitating the sample solution by applying a light vibration to the container; and
an agitation by pipetting means for agitating the sample solution by pipetting the sample solution in the container.
